(19) United States
(12) Patent Application Publication
(10) Pub. No.: US 2026/0273109 A1
(43) Pub. Date: Sep. 17, 2026

(54) COMBINATION FOR AND METHOD OF SUPPRESSING SARCOMA OR CARCINOMA MALIGNANCIES

(71) Applicant: ISM Biosciences LLC, Miami, FL (US)

(72) Inventors: Max Temnik, Miami, FL (US); Victor Gurin, Richmond, VA (US); Sergey Gurin, Richmond, VA (US)

(73) Assignee: ISM Biosciences LLC, Miami, FL (US)

(21) Appl. No.: 19/076,037

(22) Filed: Mar. 11, 2025

(51) Int. Cl. A61K 51/04; A61K 51/12; A61P 35/00

(52) U.S. Cl. CPC A61K 51/0406; A61K 51/121; A61P 35/00; A61K 2121/00

(57) ABSTRACT

A method for treating carcinoma and sarcoma tumors includes administration of a composition including ⁶⁴Znₑ(Asp) or ⁶³Cuₑ(Asp) in a therapeutically effective amount. The method may be used once a day or more than once a day. Other identified amino acids may be bound to ⁶⁴Znₑ and ⁶³Cuₑ instead of L-Aspartate (Asp), provided that such amino acids are enriched for the L-enantiomer to at least 90%. The composition is suitable for a parenteral injection or infusion. The method may be used for suppressing growth of carcinoma and sarcoma, including preventing the development of metastases.

### FIELD

The present disclosure relates to methods and compositions for treating carcinoma and sarcoma tumors using isotopically modified analogue of zinc aspartate and other isotopically modified pharmaceuticals suitable for infusion or injection.

### BACKGROUND

Carcinoma is a type of cancer that originates in epithelial tissues, which line the internal and external surfaces of the body. It is the most common form of cancer, accounting for 80-90% of all cancer diagnoses. Carcinomas are staged using the TNM system, which considers tumor size, lymph node involvement, and metastasis. Treatment options vary depending on the type, location, and stage of the carcinoma but may include surgery, radiation therapy, chemotherapy, and targeted therapies. With the exception of radiation therapy which uses enriched radioactive isotopes, these standards of care treatment methods use isotopically natural chemical elements. Although significant advances have been made in treating various types of carcinomas, and some patients can achieve long-term remissions, there is no universal cure for carcinoma cancers.

Sarcoma is a rare type of cancer that develops in connective tissues of the body. Treatment options vary depending on the type, location, and stage of the carcinoma but may include surgery, radiation therapy, chemotherapy, and targeted therapies. With the exception of radiation therapy which uses enriched radioactive isotopes, these standards of care treatment methods use isotopically natural chemical elements. Although significant advances have been made in treating various types of carcinomas, and some patients can achieve long-term remissions, there is no universal cure for sarcoma cancers.

The treatment options for carcinoma and sarcoma malignancies are still limited.

### SUMMARY

In one aspect, a method is provided of suppressing tumor growth of carcinoma or sarcoma malignancy in a patient in need thereof including administering a pharmaceutical composition to said patient, the composition including a therapeutically effective amount of ⁶⁴Znₑ(Asp), wherein from 3.8 g/kg patient weight/day to 75 mg/kg patient weight/day of ⁶⁴Znₑ(Asp) is administered to said patient and wherein the ⁶⁴Znₑ is at least 99% ⁶⁴Zn.

In another aspect, a method is provided of suppressing tumor growth of carcinoma or sarcoma malignancy in a patient in need thereof including administering a pharmaceutical composition to said patient, the composition including a therapeutically effective amount of ⁶³Cuₑ(Asp), wherein from 3.4 g/kg patient weight/day to 45 mg/kg patient weight/day of ⁶³Cuₑ(Asp) is administered to said patient and wherein the ⁶³Cuₑ is at least 99% ⁶³Cu.

### BRIEF DESCRIPTION OF DRAWINGS

FIG. 1 illustrates isotopic fractionation of zinc isotopes in breast cancer tissues, showing that breast carcinoma tumors are enriched with heavier zinc isotopes relative to natural isotopic abundance of zinc.

FIG. 2 illustrates the cytotoxic effect of ⁶⁴Zn-Aspartate, ⁶⁴Zn-Sulfate, and ⁶⁴Zn-Citrate in Lewis carcinoma cells.

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should be considered to include the end points 5 and 10.

The feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein shall have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “chemical element” refers to any substance that cannot be decomposed into simpler substances by chemical processes. Elements are the fundamental materials of which all matter is composed.

The term “isotope” refers to a variant of an atom of a chemical element that differs by the number of neutrons in its nucleus and has a different atomic mass. According to the proton-neutron model developed by D. I. Ivanenko and W. Heisenberg (1932), atoms of all chemical elements consist of three types of elementary particles: positively charged protons, negatively charged electrons, and neutrons that have no charge. The number of protons p in the nucleus determines the atomic number Z of the chemical element in Mendeleev's periodic table. The proton and the neutron, which have a common name—nucleons—have almost identical weight. The mass of the neutron (1.00866 amu) is somewhat greater than the proton mass (1.00727 amu). The electron mass is much smaller than that of the nucleons (for example, the proton-to-electron mass ratio is 1836.13). Therefore, the mass of the atom is concentrated in its nucleus. Hence, the mass number of the atom A is connected with the atomic number by a simple relation A=p+n=Z+n, where n is the number of neutrons in the nucleus of an atom. The number of protons in the nucleus of an atom uniquely determines the position of an element in the periodic table of the elements. Furthermore, the number of protons determines the number of electrons present in a neutral atom thus determining the chemical properties of this atom. However, atoms with the same atomic number Z (and hence the number of protons p) may have different neutron numbers n. Thus, atoms with different atomic mass numbers may occupy the same position on the periodic table. Chemical elements having the same atomic number but a different atomic mass are known as isotopes.

Stable isotopes are a known matter. Although radioactive isotopes have been used in medicine for diagnostics and radiation therapies, the use of enriched stable isotopes has been limited to the applications in diagnostics, clinical pharmacology, and metabolic studies, all of which have used stable isotopes in their naturally abundant ratios. In diagnostics, stable isotopes have been used as tracers for dynamically assessing in vivo metabolism. In research, stable isotopes have been used in evaluating bioavailability and the release profile of drug products and drug delivery systems. In the assessment of drug pharmacology, stable isotopes have been used for the determination of a drug's pharmacokinetic profile, mechanism of action, and potential toxicity or adverse effects. In precision medicine, stable isotopes have recently started to be used monitoring drug treatment effects and conducting clinical toxicology studies. In nutrition, stable isotopes have been used to assess body composition, energy expenditure, protein turnover, food safety and metabolic profiles. However, all of these prior art techniques and technologies have used stable isotopes in their naturally occurring isotopic ratios.

The term “natural abundance” of an isotope refers to the fraction of the total amount of the corresponding element that the isotope represents, on a mole-fraction basis (that is, not, for example, on a mass basis). For example, ⁶⁴Zn had a natural abundance fraction of 48.63%, meaning that 48.63% of Zn atoms on earth are the isotope ⁶⁴Zn. When a composition is “enriched” for a certain isotope, the abundance of the isotope in the composition is greater than the isotope's natural abundance. For ⁶⁴Zn, a composition in which ⁶⁴Zn constitutes more than 48.63% of the total Zn in the composition, on a mole-fraction basis, would be “enriched” for ⁶⁴Zn. Throughout this application, a subscript “e” following a light isotope chemical symbol or element name indicates that the designated element is enriched for that isotope. For example, ⁶⁴Zn refers to the light isotope zinc-64, whereas ⁶⁴Znₑ refers to zinc that is enriched for zinc-64. Thus, “⁶⁴Znₑ aspartate,” for example, refers to zinc aspartate in which the zinc is enriched for zinc-64.

In some embodiments, ⁶⁴Znₑ(Asp), or zinc-64 aspartate, has a chemical formula —(C₄H₅O₄N)₂⁶⁴Znₑ, with 2 aspartic acid molecules. In other embodiments, ⁶⁴Znₑ (Asp) has a chemical formula

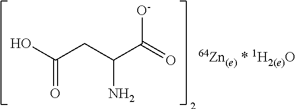

In other embodiments, ⁶⁴Znₑ (Asp), or protium-/¹⁶O-enriched zinc-64 aspartate, has a chemical formula

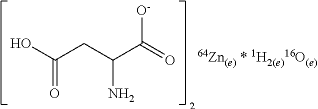

The term “relative isotopic fraction” refers to a measure of the proportion of one isotope of a chemical element relative to another isotope of the sample of the same element, typically expressed in comparison to a standard reference. For example, the isotopic fraction of ⁶⁴Zn atoms of naturally occurring zinc is 48.6%.

The term “enriched” refers to the process of increasing the proportional fraction of a specific isotope within a mixture of isotopes. The enrichment process exploits the differences in physical and/or chemical properties between isotopes of the same element to separate and concentrate one isotope from the others. The term “isotopically modified” refers to the process of enriching an identified isotope. Hence, terms “enrichment” and “isotopic modification” essentially refer to the same concept. Isotope enrichment is also a known process of isotopic modification that refers to increasing the fraction of a specific isotope in a mixture of isotopes in a chemical element. An enrichment of light isotopes automatically results in a depletion of heavy isotopes of the same chemical element, and vice versa.

The enriched stable isotopes of hydrogen (H), carbon (C), nitrogen (N), and oxygen (O) in both medicine and pharmaceutical applications have been used. These enriched isotopes were used for various purposes, including drug development. For instance, isotopes ¹³C and ¹⁵N have been used in clinical medicine and biological studies. These isotopes are particularly valuable in the development of diagnostic tests, such as the ¹³C urea breath test for detecting Helicobacter pylori infections. Also, the chemical compounds labeled with highly enriched ¹³C are used in breath tests for diagnosing liver and intestine diseases. Stable isotopes ²H, ³H, ¹³C, N, and ¹⁸O are heavy stable isotopes.

The term “therapeutic efficacy” refers to the ability of a medical intervention, such as a pharmaceutical composition or treatment method, to produce a beneficial effect under ideal and controlled circumstances. It is a critical concept in pharmacology and medicine, used to evaluate the potential of new treatments.

The terms “effective amount,” “prophylactically effective amount,” or “therapeutically effective amount” refer to an amount of an agent or composition that provides a beneficial effect or favorable result to a subject, or alternatively, an amount of an agent or composition that exhibits the desired in vivo or in vitro activity. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations. “Effective amount,” “prophylactically effective amount,” or “therapeutically effective amount” can refer to an amount of an agent or composition that provides a biological, therapeutic, and/or prophylactic result.

For any composition, in some embodiments, an effective amount can be first estimated either in accordance with cell culture assays or using animal models, typically mice, rats, guinea pigs, rabbits, dogs or pigs. An animal model may be used to determine an appropriate concentration range and route of administration. Such information can then be used to determine appropriate doses and routes of administration for humans. When calculating a human equivalent dose, it is recommended to use a conversion table given in the Guidance for Industry and the Reviewers document (2002, US Food and Drug Administration, Rockville, MD, USA). An effective daily dose is generally 0.01 mg/kg patient weight to 2000 mg/kg patient weight of an active agent, preferably 0.05 mg/kg patient weight to 500 mg/kg patient weight of an active agent. An exact effective dose will depend on the severity of the disease, patient's general state of health, age, body weight and sex, nutrition, time and frequency of administration, combination(s) of medicines, response sensitivity and tolerance/response to administration and other factors that will be taken into account by a person skilled in the art when determining the dosage and route of administration for a particular patient based on his/her knowledge of the art. Such dose may be determined by conducting routine experiments and at the physician's discretion. Effective doses will also vary depending on the possibility of their combined use with other therapeutic procedures, such as the use of other agents.

The term “nascent form” refers to an isotope that is freshly formed, meaning it has just been created and is in its most reactive state, often with a higher potential for chemical reactions due to its immediate availability and lack of interaction with other molecules; essentially, it's an isotope in the moment of its formation, before it has had a chance to stabilize or bond with anything else.

The term “colloidal form” refers to a specific isotope of an element that exists as nanoparticles suspended in a liquid, forming a colloid; essentially, the isotope is dispersed in a medium as very small, stable particles that are too small to settle out readily, creating a uniform mixture.

The term “elemental form” refers to a specific variant of a chemical element, where most atoms of that element have the same number of protons and the same number of neutrons, essentially meaning they are the same isotopes of the same element, existing in their pure state as a chemical element.

The term “atomic form” refers to a single atom of that element.

The term “molecule” is known in the art. Briefly, it refers to an electrically neutral group of two or more atoms held together by chemical bonds. Molecules form the smallest identifiable unit of a chemical compound that retains the composition and chemical properties of that compound. Examples include carbohydrates, lipids, and ribonucleic acid.

The term “suppressing tumor growth” as used herein with respect to a medical condition such as any type or kind of sarcoma or carcinoma refers to lessening severity and/or consequences of the condition, slowing progression of the condition, reversing pathology of the condition, and/or curing the condition.

The term “preventing” as used herein with respect to a medical condition, such as any type or kind of sarcoma or carcinoma metastasis refers to preventing the development of metastasis, diminishing the severity of metastasis, lessening consequences of metastasis, and/or slowing progression of metastasis.

The terms “patient” and “subject” are interchangeable terms and may refer to a human patient/subject, a dog, a cat, a non-human primate, and other mammals.

The term “carcinoma” refers to the category of cancers that originate in epithelial tissues. The major subtypes of carcinoma include adenocarcinoma, squamous cell carcinoma, and basal cell carcinoma. There is no specific number given for the total kinds of carcinomas presently known. There are numerous specific types of carcinomas based on their location and cellular characteristics. The classification of cancers, including carcinomas, is continually evolving as research advances our understanding of cancer biology and genetics.

The term “sarcoma” refers to the category of cancers that originate in connective tissues of the body, such as bones, muscles, cartilage, tendons, blood vessels, fat, and nerves. Two main types of sarcomas are soft tissue sarcomas and bone sarcomas. Within these broad categories, sarcomas are further classified based on the specific type of tissue they originate from, such as muscle, fat, blood vessels, nerves, and connective tissues. The classification of sarcomas is continually evolving as research advances our understanding of cancer biology and genetics. Therefore, the exact number of recognized subtypes may change over time as new discoveries are made and classifications are refined.

Isotopically natural chemical elements have been historically used for making various organic molecules and pharmaceutical compositions. Enriched medical radioisotopes have been used in pharmaceutical industry since the early 1930s, playing an important role in advancing nuclear medicine and enabling the development of more effective therapeutic radiopharmaceuticals. One of the earliest recorded uses was by John Lawrence, who in 1936 used phosphorus-32, a radioactive isotope, to treat leukemia. This marked the first clinical therapeutic application of an artificially enriched radionuclide.

The development of organic compounds has traditionally relied on naturally occurring isotopes of chemical elements. The use of enriched stable isotopes in pharmaceuticals as part of organic molecules commenced with the discovery of deuterium by Harold Urey in 1932. For the first several decades, the focus was on understanding biological and chemical processes, leveraging the ability of stable isotopes like deuterium (Rita Maria Concetta Di Martino, 2023) and tritium to be detected through mass spectrometry and radioactivity measurements, respectively. Despite tritium being a radioactive isotope, its long half-life and the stable nature of deuterium allowed for safe handling and application in various biomedical areas. The concept of deuteration (substituting a hydrogen atom with deuterium atom) emerged as a significant advancement in drug discovery in the following decades. This innovative approach emerged with the development of deutetrabenazine (U.S. Pat. No. 8,524,733) approved by U.S. FDA in 2017 and deucravacitinib (WO 2018/183656) approved in 2022. Both deutetrabenazine and deucravacitinib use isotopically modified hydrogen ¹H. These breakthroughs marked the beginning of a new era in pharmaceutical design, focusing on the isotopic modification of stable isotopes.

Zinc aspartate is a form of zinc compound that combines isotopically natural zinc with aspartic acid. It is commonly used to treat zinc deficiency and may offer several potential health benefits. Isotopically natural zinc has five stable isotopes, with their percentages in nature in parenthesis: ⁶⁴Zn (48.63%), ⁶⁶Zn (27.90%), ⁶⁷Zn (4.10%), ⁶⁸Zn (18.75%), and ⁷⁰Zn (0.62%).

The ⁶⁴Znₑ aspartate disclosed herein is isotopically modified by enrichment of ⁶⁴Zn atoms to at least 99%, and, in certain embodiments, enrichment of aspartate for L-enantiomer to at least 90%.

Copper aspartate is used as a dietary supplement to provide this essential mineral for various bodily functions. Isotopically natural copper aspirinate, a related compound, has shown promise in treating rheumatoid arthritis. Copper has two stable isotopes that occur naturally, with their percentages in nature in parenthesis: ⁶³Cu (69.17%) and ⁶⁵Cu (30.83%).

The ⁶³Cuₑ aspartate disclosed herein is isotopically modified by enrichment of ⁶³Cu atoms to at least 99%, and, in certain embodiments, enrichment of aspartate for L-enantiomer to at least 90%.

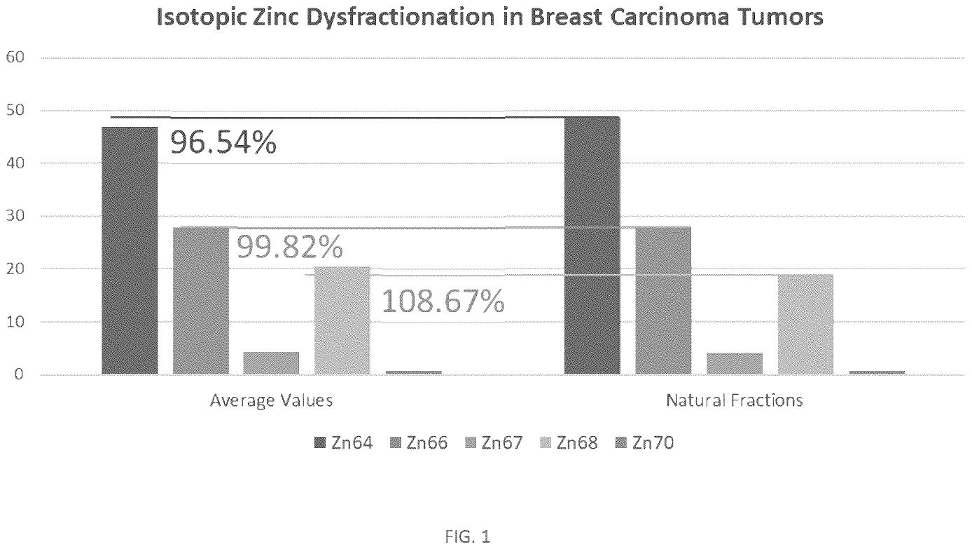

Recently, a number of works have demonstrated that isotopic composition of tissues and organs may serve as a diagnostic marker. Furthermore, studies demonstrate that sarcoma and carcinoma cancer cells exhibit isotopic fractionation of zinc and copper. The data on isotopic fractionation of zinc and copper are conflicting and often report the opposite results. Clinical samples of breast cancer tissue showed lighter Zn isotopic compositions (δ66Zn) relative to healthy tissue. Breast carcinoma tumors were found to have a significantly lighter Zn isotopic composition than blood, serum and healthy breast tissue (Schilling et al., Front Med (Lausanne). 2022 Jan. 20; 8:746532.) However, data we obtained shows the opposite results (see FIG. 1.) Prostate cancer patients show lower urinary δ66Zn values compared to healthy controls. The δ⁶⁶Zn values for pancreatic adenocarcinoma, which makes ~90% of all pancreatic cancers, range between −0.33‰ to +0.15‰, with a median value of −0.15‰. Healthy controls tend to have higher δ66Zn values, ranging between −0.26‰ to 0.67‰, with a median value of +0.02‰. The δ⁶⁶Zn notation represents the relative difference in the ⁶⁶Zn/⁶⁴Zn ratio between a sample and a standard, typically expressed in parts per thousand (‰). Hence, a decreased δ66Zn value refers to the increased ⁶⁴Zn fraction. The same is true for copper: an increased δ⁶⁵Cu value refers to the decreased ⁶³Cu fraction.

Further, certain patents and patent applications discuss the use of isotopically enriched compositions for therapeutic use. See, e.g., U.S. Pat. Nos. 9,861,659; 10,183,041, and 12,138,322.

Carcinoma is a type of cancer that originates in epithelial tissues, which line the internal and external surfaces of the body. It is the most common form of cancer, accounting for 80-90% of all cancer diagnoses. Carcinomas are staged using the TNM system, which considers tumor size, lymph node involvement, and metastasis. Treatment options vary depending on the type, location, and stage of the carcinoma but may include surgery, radiation therapy, chemotherapy, and targeted therapies. With the exception of radiation therapy which uses enriched radioactive isotopes, these standards of care treatment methods use isotopically natural chemical elements. Although significant advances have been made in treating various types of carcinomas, and some patients can achieve long-term remissions, there is no universal cure for carcinoma cancers.

Sarcoma is a rare type of cancer that develops in connective tissues of the body. Treatment options vary depending on the type, location, and stage of the carcinoma but may include surgery, radiation therapy, chemotherapy, and targeted therapies. With the exception of radiation therapy which uses enriched radioactive isotopes, these standards of care treatment methods use isotopically natural chemical elements. Although significant advances have been made in treating various types of carcinomas, and some patients can achieve long-term remissions, there is no universal cure for sarcoma cancers.

As used herein, unless otherwise indicated, X % ⁶⁴Zn means that, out of 100 zinc atoms, X is ⁶⁴Zn. For example, in a composition that includes zinc that is 99% ⁶⁴Zn, 99% of zinc atoms are ⁶⁴Zn. Likewise, X % ⁶³Cu means that, out of 100 copper atoms, X is ⁶³Cu. For example, in a composition that includes copper that is 90% ⁶³Cu, 90% of copper atoms are ⁶³Cu. Unless otherwise indicated, the term “⁶⁴Znₑ” is used herein as shorthand for “⁶⁴Zn-enriched zinc” and term “⁶³Cuₑ” is used herein as shorthand for “⁶³Cu-enriched copper”.

In one aspect, this disclosure provides a method of suppressing tumor growth of carcinoma or sarcoma malignancy in a patient in need thereof including administering a pharmaceutical composition to said patient, the composition including a therapeutically effective amount of ⁶⁴Znₑ(Asp), wherein from 3.8 g/kg patient weight/day to 75 mg/kg patient weight/day of ⁶⁴Znₑ(Asp) is administered to said patient and wherein the ⁶⁴Znₑ is at least 99% ⁶⁴Zn. In some embodiments, from 3.8 g/kg patient weight/day to 75 mg/kg patient weight/day of ⁶⁴Znₑ(Asp) is administered to said patient intratumorally or intravenously and wherein the ⁶⁴Znₑ is at least 99% ⁶⁴Zn. In some embodiments, the ⁶⁴Znₑ(Asp) is in an aqueous solution. In some embodiments, the composition includes deuterium-depleted water. In some embodiments, the ⁶⁴Znₑ(Asp)₂ comprises 2 molecules of aspartic acid per each atom of ⁶⁴Znₑ. In some embodiments, the composition is administered more often than once per day. In some embodiments, the composition includes water in which oxygen is at least 99.8% ¹⁶O or at least 0.05% ¹⁷O. In some embodiments, the aspartic acid is enriched for L-enantiomer to at least 90%. In some embodiments, the aspartic acid is replaced by glutamic acid or glutamate, wherein the glutamic acid or glutamate is enriched for the L-enantiomer to at least 90%. In some embodiments, the aspartic acid is replaced by proline, alanine, or tyrosine, or a combination thereof, wherein any of proline, alanine, or tyrosine is enriched for L-enantiomer to at least 90%. In some embodiments, the composition is used for preventing the development of metastases. In some embodiments, the aspartic acid is replaced by at least of one of sulfuric acid or citric acid. In some embodiments, the aspartic acid is replaced by at least one of indole-3-carbinol or isoleucine enriched for L-enantiomer to at least 90%.

In another aspect, this disclosure provides a method of suppressing tumor growth of carcinoma or sarcoma malignancy in a patient in need thereof including administering a pharmaceutical composition to said patient, the composition including a therapeutically effective amount of ⁶³Cuₑ(Asp), wherein from 3.4 g/kg patient weight/day to 45 mg/kg patient weight/day of ⁶³Cuₑ(Asp) is administered to said patient and wherein the ⁶³Cuₑ is at least 99% ⁶³Cu. In some embodiments, from 3.4 g/kg patient weight/day to 45 mg/kg patient weight/day of ⁶³Cuₑ(Asp) is administered to said patient intratumorally or intravenously and wherein the ⁶³Cuₑ is at least 99% ⁶³Cu. In some embodiments, the ⁶³Cuₑ(Asp) is in an aqueous solution. In some embodiments, the composition includes deuterium-depleted water. In some embodiments, the ⁶³Cuₑ(Asp)₂ includes 2 molecules of aspartic acid per each atom of ⁶³Cuₑ. In some embodiments, the composition is administered more often than once per day. In some embodiments, the composition includes water in which oxygen is at least 99.8% ¹⁶O or at least 0.05% ¹⁷O. In some embodiments, the aspartic acid is enriched for L-enantiomer to at least 90%. In some embodiments, the aspartic acid is replaced by glutamic acid or glutamate, wherein the glutamic acid or glutamate enriched for L-enantiomer to at least 90%. In some embodiments, the aspartic acid is replaced by proline, wherein the proline is enriched for L-enantiomer to at least 90%. In some embodiments, the composition is used for preventing the development of metastases. In some embodiments, the aspartic acid is replaced by at least of one of sulfuric acid or citric acid. In some embodiments, the aspartic acid is replaced by at least one of indole-3-carbinol or isoleucine enriched for L-enantiomer to at least 90%.

The typical dosage of isotopically natural zinc aspartate ranges from 10-30 mg of elemental zinc per day regardless of the person's body weight. Hence, such dose ranges from 0.125 mg/kg patient weight to 0.375 mg/kg of patient weight for an average adult weighing 80 kg. In certain embodiments, the therapeutically or prophylactically effective dose of isotopically modified ⁶⁴Znₑ(Asp) disclosed herein ranges from 3.8 g/kg patient weight per day to 75 mg/kg patient weight per day, or up to 6 g for the patient weighing 80 kg. The isotopically natural zinc aspartate used in prior art is administered orally. In certain embodiments, the isotopically modified ⁶⁴Znₑ(Asp) disclosed herein is administered by routes of parenteral injections or infusions. In certain embodiments, the isotopically modified ⁶⁴Znₑ(Asp) disclosed herein is administered intratumorally, parenterally, or intravenously.

The typical dosage of isotopically natural copper aspartate ranges from 8-11 mg per day of elemental zinc per day regardless of the person's body weight. Hence, such dose ranges from 0.1 mg/kg patient weight to 0.1375 mg/kg of patient weight for an average adult weighing 80 kg. In certain embodiments, the therapeutically or prophylactically effective dose of isotopically modified ⁶³Cuₑ(Asp) disclosed herein ranges from 3.4 g/kg patient weight per day to 45 mg/kg patient weight per day, or up to 3.6 g for the patient weighing 80 kg. The isotopically natural copper aspartate used in prior art is administered orally. In certain embodiments, the isotopically modified ⁶⁴Cuₑ(Asp) disclosed herein is administered by routes of parenteral injections or infusions. In certain embodiments, the isotopically modified ⁶⁴Cuₑ(Asp) disclosed herein is administered intratumorally, parenterally, or intravenously.

In some embodiments, the ⁶⁴Znₑ or the ⁶³Cuₑ concentration exceeds 80.4 μM.

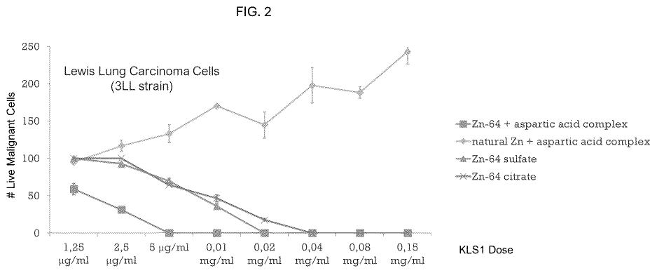

FIG. 2 illustrates the cytotoxic effect of ⁶⁴Zn-Aspartate, ⁶⁴Zn-Sulfate, and ⁶⁴Zn-Citrate in Lewis carcinoma cells.

The disclosed composition can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the disclosed compositions comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method of suppressing tumor growth of carcinoma or sarcoma malignancy in a patient in need thereof including administering a pharmaceutical composition to said patient, the composition including a therapeutically effective amount of ⁶⁴Znₑ(Asp), wherein from 3.8 μg/kg patient weight/day to 75 mg/kg patient weight/day of ⁶⁴Znₑ(Asp) is administered to said patient intratumorally or intravenously and wherein the ⁶⁴Znₑ is at least 99% ⁶⁴Zn, wherein the composition comprises 2 molecules of aspartic acid per each atom of ⁶⁴Znₑ.

2. The method of claim 1, wherein the ⁶⁴Znₑ(Asp) composition is in an aqueous solution.

3. The method of claim 1, wherein the composition includes deuterium-depleted water.

4. (canceled)

5. The method of claim 1, wherein the composition is administered more often than once per day.

6. The method of claim 1, wherein the composition includes water in which oxygen is at least 99.8% ¹⁶O or at least 0.05% ¹⁷O.

7. The method of claim 1, wherein the aspartic acid is enriched for L-enantiomer to at least 90%.

8. The method of claim 1, wherein the aspartic acid is replaced by sulfuric acid or citric acid.

9. The method of claim 1, wherein the aspartic acid is replaced by indole-3-carbinol or isoleucine enriched for L-enantiomer to at least 90%.

10-20. (canceled)

32. A method of suppressing tumor growth of carcinoma or sarcoma malignancy in a patient in need thereof including administering a pharmaceutical composition to said patient, the composition including a therapeutically effective amount of ⁶⁴Znₑ(Asp), wherein from 3.8 μg/kg patient weight/day to 75 mg/kg patient weight/day of ⁶⁴Znₑ(Asp) is administered to said patient intratumorally or intravenously and wherein the ⁶⁴Znₑ is at least 99% ⁶⁴Zn, wherein:
the composition comprises 2 molecules of aspartic acid per each atom of ⁶⁴Znₑ;
the ⁶⁴Znₑ(Asp) composition is in an aqueous solution;
the composition includes deuterium-depleted water.

33. The method of claim 32, wherein the aspartic acid is replaced by sulfuric acid or citric acid.

34. The method of claim 32, wherein the aspartic acid is replaced by indole-3-carbinol or isoleucine enriched for L-enantiomer to at least 90%.
